(19) United States
(12) Patent Application Publication
(10) Pub. No.: US 2015/0230775 A1
(43) Pub. Date: Aug. 20, 2015

(54) IMAGING APPARATUS FOR DIAGNOSIS, INFORMATION PROCESSING APPARATUS, AND CONTROL METHOD THEREFOR

(71) Applicant: TERUMO KABUSHIKI KAISHA, Tokyo (JP)

(72) Inventor: Youhei KOBAYASHI, Hadano-City (JP)

(73) Assignee: TERUMO KABUSHIKI KAISHA, Tokyo (JP)

(21) Appl. No.: 14/704,315

(22) Filed: May 5, 2015

Related U.S. Application Data
(63) Continuation of application No. PCT/JP2012/007098, filed on Nov. 6, 2012.

(51) Int. Cl. A61B 8/00; G06T 3/40; G06T 7/00; G06T 11/60; A61B 5/00; A61B 8/12

(52) U.S. Cl. CPC A61B 8/4416; A61B 5/0066; A61B 8/12; A61B 8/54; A61B 8/463; G06T 7/0012; G06T 11/60; G06T 3/40; G06T 2200/24; G06T 2207/10004; G06T 2207/10101; G06T 2207/10132; G06T 2207/30004

(57) ABSTRACT

An imaging apparatus is disclosed for diagnosis, which can display a tomographic image of biological tissues using ultrasound waves and light, and wherein the imaging apparatus can accurately perform diagnosis on biological tissues, in which while a user is suppressing a visual loss between an ultrasound cross-sectional image and an optical cross-sectional image, the user can check his or her interest site in the biological tissues by using both the ultrasound cross-sectional image and the optical cross-sectional image, without changing the user's viewpoint position. Therefore, in a magnifier mode, a circular frame indicating a magnifier whose position can be freely changed by the user inside an image display region is displayed inside the image display region. Then, an IVUS cross-sectional image is displayed in a region outside the circular frame within the image display region, and a partial image of an OCT cross-sectional image is displayed inside the circular frame.

### CROSS-REFERENCES TO RELATED APPLICATIONS

This application is a continuation of International Application No. PCT/JP2012/007098 filed on Nov. 6, 2012, the entire content of which is incorporated herein by reference.

### TECHNICAL FIELD

The present disclosure generally relates to an imaging apparatus for diagnosis, which can display a tomographic image of biological tissues using ultrasound waves and light, an information processing apparatus, and a control method therefor.

### BACKGROUND DISCUSSION

An imaging apparatus for diagnosis has been used for diagnosis of arteriosclerosis, preoperative diagnosis in performing endovascular treatment using a high-performance catheter such as a balloon catheter, a stent or the like, or for confirmation of postoperative results.

The imaging apparatus for diagnosis can include an intravascular ultrasound (IVUS) diagnosis apparatus and an optical coherence tomography (OCT) diagnosis apparatus, which respectively have different characteristics.

In addition, in recent years, an imaging apparatus for diagnosis (imaging apparatus for diagnosis which can include an ultrasound transceiver capable of transmitting and receiving ultrasound waves and an optical transceiver capable of transmitting and receiving light) which has an IVUS function and an OCT function in combination has also been proposed (for example, refer to JP-A-11-56752 and JP-A-2006-204430). According to this imaging apparatus for diagnosis, single scanning can generate both a cross-sectional image utilizing IVUS characteristics, which can enable measurement for a very deep region and a cross-sectional image utilizing OCT characteristics, which can enable high-resolution measurement.

As described above, the cross-sectional image at the same location inside the blood vessel can be generated by using both the IVUS function and the OCT function. According to the OCT cross-sectional image, a high-resolution image can be obtained for relatively shallow tissues, but an image for the deeper tissues cannot be obtained in some aspects. According to the IVUS cross-sectional image, an image including relatively deep tissues can be conveniently obtained, but the image cannot be an image with as high a resolution as the OCT cross-sectional image in some aspects. That is, it is considered that these two types of the cross-sectional image are in a relationship to compensate for each other.

According to an up-to-date display method, these two types of the cross-sectional image can be displayed side by side, or a single synthesized image can be generated and displayed by synthesizing these two types of the cross-sectional image.

In the former case, it can be necessary for a user to visually compare two types of the cross-sectional image, which are located at a distance from one another on a screen. Accordingly, the user has no choice but to imagine conditions of a lesion in his or her head.

In the latter case, the burden in diagnosis on the user can be less since the user does not need to move his or her viewpoint that much (refer to JP-T-2010-516304). However, according to a general method for synthesizing two cross-sectional images, an average value of pixel values of the two cross-sectional images can be calculated so that the average value represents a value of one pixel of the synthesized image. Thus, for example, characteristics belonging to the OCT cross-sectional image in the synthesized image can be reduced to half of the characteristics of the original OCT cross-sectional image, which means that half of the information belonging to the original OCT cross-sectional image is lost. This is also true in the IVUS cross-sectional image. Since the synthesized image is displayed, the user needs to stop the display of the synthesized image for the time being, for example, in order to view a pure OCT image excluding the IVUS cross-sectional image. Consequently, the operation can become complicated.

In addition, JP-T-2010-516304 discloses a technology in which a boundary line is set inside one image between the OCT cross-sectional image and the IVUS image so as to display the other cross-sectional image inside an outline thereof. According to this configuration, an advantageous effect can be that two images can be compared with each other without changing a user's viewpoint.

However, according to the technology disclosed in JP-T-2010-516304, another problem could occur. For example, where a region indicated by the boundary line in the OCT cross-sectional image is set to display the IVUS cross-sectional image therein, the OCT cross-sectional image hidden in the IVUS cross-sectional image cannot be checked. Consequently, a problem can occur in that a region for displaying only the OCT cross-sectional image can be separately required in order to check a portion hidden in the IVUS cross-sectional image.

### SUMMARY

The present disclosure is made in view of the problems disclosed above, a technology is disclosed for accurately performing diagnosis on biological tissues, in which while a user is suppressing a visual loss between an ultrasound cross-sectional image and an optical cross-sectional image, the user can check his or her interest site in the biological tissues by using both the ultrasound cross-sectional image and the optical cross-sectional image, even without changing the user's viewpoint position.

In accordance with an exemplary embodiment, an imaging apparatus for diagnosis is disclosed, which can rotatably and detachably hold a probe having a transceiver in which an ultrasound transceiver for transmitting and receiving ultrasound waves and an optical transceiver for transmitting and receiving light are arranged, and which can generate an ultrasound cross-sectional image and an optical cross-sectional image of biological tissues by using the reflected waves reflected from biological tissue and received by the ultrasound transceiver and the reflected light reflected from the biological tissue and received by the optical transceiver. The apparatus can include a display means for displaying an image display region for displaying a cross-sectional image, and a user interface including a frame which is located inside the image display region and whose display position is freely changed in accordance with a position instructed by a user, and a display control means for changing the display position of the frame in accordance with the user's instruction, for displaying a first cross-sectional image, one image of the biological tissues between the ultrasound cross-sectional image and the optical cross-sectional image which correspond to each other, in a region outside the frame within the image display region, and for displaying a second cross-sectional image, the other image between the ultrasound cross-sectional image and the optical cross-sectional image, inside the frame.

In accordance with an exemplary embodiment, an information processing apparatus is disclosed which can display an ultrasound cross-sectional image and an optical cross-sectional image which can be obtained by an imaging apparatus for diagnosis for generating the ultrasound cross-sectional image and the optical cross-sectional image. The apparatus can include a display means for displaying an image display region for displaying a cross-sectional image, and a user interface including a frame which is located inside the image display region and whose display position is freely changed in accordance with a position instructed by a user, and a display control means for changing the display position of the frame in accordance with the user's instruction, for displaying a first cross-sectional image, one image of the biological tissues between the ultrasound cross-sectional image and the optical cross-sectional image which correspond to each other, in a region outside the frame within the image display region, and for displaying a second cross-sectional image, the other image between the ultrasound cross-sectional image and the optical cross-sectional image, inside the frame.

According to the present disclosure, a technology is disclosed, which can help a user to perform diagnosis on both an ultrasound cross-sectional image and an optical cross-sectional image without changing the user's viewpoint position, and which can help the user to easily check a lesion located at the user's desired position by using any one of the cross-sectional images.

Other characteristics and advantages of the present disclosure will become apparent from the following description made with reference to the accompanying drawings. In the accompanying drawings, the same reference numerals are given to the same or similar configuration elements.

### BRIEF DESCRIPTION OF THE DRAWINGS

The accompanying drawings are incorporated in the description, configure a part of the description, represent exemplary embodiments of the present disclosure, and are used to describe principles of the present disclosure together with the description.

FIG. 1 is a view illustrating an external configuration of an imaging apparatus for diagnosis according to an exemplary embodiment of the present disclosure.

FIG. 2 is a view illustrating an overall configuration of a probe unit and a cross-sectional configuration of a distal end portion.

FIG. 3A is a diagram illustrating a cross-sectional configuration of an imaging core.

FIG. 3B is a cross-sectional view taken along a plane substantially orthogonal to a rotation center axis at an ultrasonic transmitting and receiving position.

FIG. 3C is a cross-sectional view taken along a plane substantially orthogonal to the rotation center axis at an optical transmitting and receiving position.

FIG. 4 is a diagram illustrating a functional configuration of the imaging apparatus for diagnosis.

FIG. 5 is a view illustrating an example of an IVUS image and an OCT image, which are built in a memory when intravascular scanning is completed.

FIG. 6 is a view illustrating an example of a user interface.

FIG. 7 is a view illustrating an example of a user interface.

FIG. 8 is a view illustrating processing content in a magnifier mode.

FIG. 9 is a flowchart illustrating the processing content in the magnifier mode.

FIG. 10 is a flowchart illustrating details in Step S916 illustrated in FIG. 9.

### DETAILED DESCRIPTION

Hereinafter, an embodiment according to the present disclosure will be described in detail with reference to the accompanying drawings.

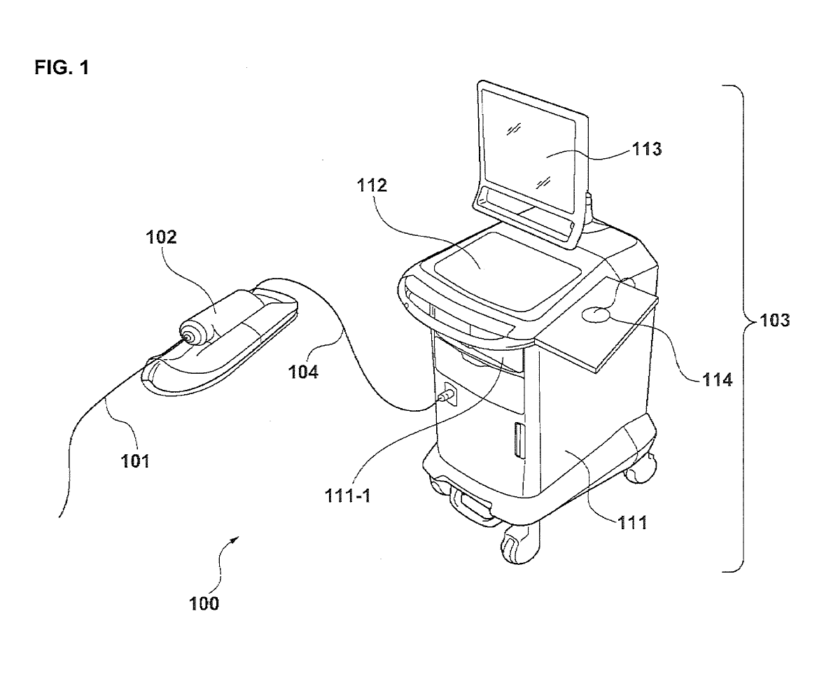

FIG. 1 is a view illustrating an external configuration of an imaging apparatus for diagnosis (imaging apparatus for diagnosis which can include an IVUS function and an OCT function) 100 according to an embodiment of the present disclosure.

As illustrated in FIG. 1, the imaging apparatus for diagnosis 100 can include a probe unit 101, a scanner and pull-back unit 102, and an operation control device 103. The scanner and pull-back unit 102 and the operation control device 103 can be connected to each other by a signal line 104 so that various signals can be transmitted.

The probe unit 101 has an internally inserted imaging core which is directly inserted into a blood vessel and can include an ultrasound transceiver which transmits ultrasound waves into the blood vessel based on a pulse signal and which receives reflected waves from the inside of the blood vessel, and an optical transceiver which continuously transmits transmitted light (measurement light) into the blood vessel and which continuously receives reflected light from the inside of the blood vessel. The imaging apparatus for diagnosis 100 measures an intravascular state by using the imaging core.

The probe unit 101 can be detachably attached to the scanner and pull-back unit 102. A motor incorporated in the scanner and pull-back unit 102 is driven, thereby regulating an intravascular operation in the axial direction and an intravascular operation in the rotation direction of the imaging core, which is internally inserted into the probe unit 101. In addition, the scanner and pull-back unit 102 acquires the reflected wave received by the ultrasound transceiver and the reflected light received by the optical transceiver, and transmits the reflected wave and the reflected light to the operation control device 103.

The operation control device 103 can include a function for inputting various setting values upon each measurement, and a function for processing data obtained by the measurement and for displaying an intravascular cross-sectional image (images of cross-sections in a direction traversing a blood vessel axis, for example, images of cross-sections perpendicular to the blood vessel axis) and longitudinal images (images of cross sections parallel to the blood vessel axis).

In the operation control device 103, the reference numeral 111 represents a main body control unit which generates ultrasound data based on the reflected waves obtained by the measurement, and which generates an ultrasound cross-sectional image by processing line data generated based on the ultrasound data. Furthermore, the main body control unit 111 generates interference light data by causing the reflected light obtained by the measurement to interfere with reference light obtained by separating the light from a light source, and generates an optical cross-sectional image by processing the generated line data based on the interference light data.

The reference numeral 111-1 represents a printer and DVD recorder, which prints a processing result in the main body control unit 111 or stores the processing result as data. The reference numeral 112 represents an operation panel, and a user inputs various setting values and indications via the operation panel 112. The reference numeral 113 represents an LCD monitor as a display device, which displays a cross-sectional image generated in the main body control unit 111. The reference numeral 114 represents a mouse serving as a pointing device (digitizer).

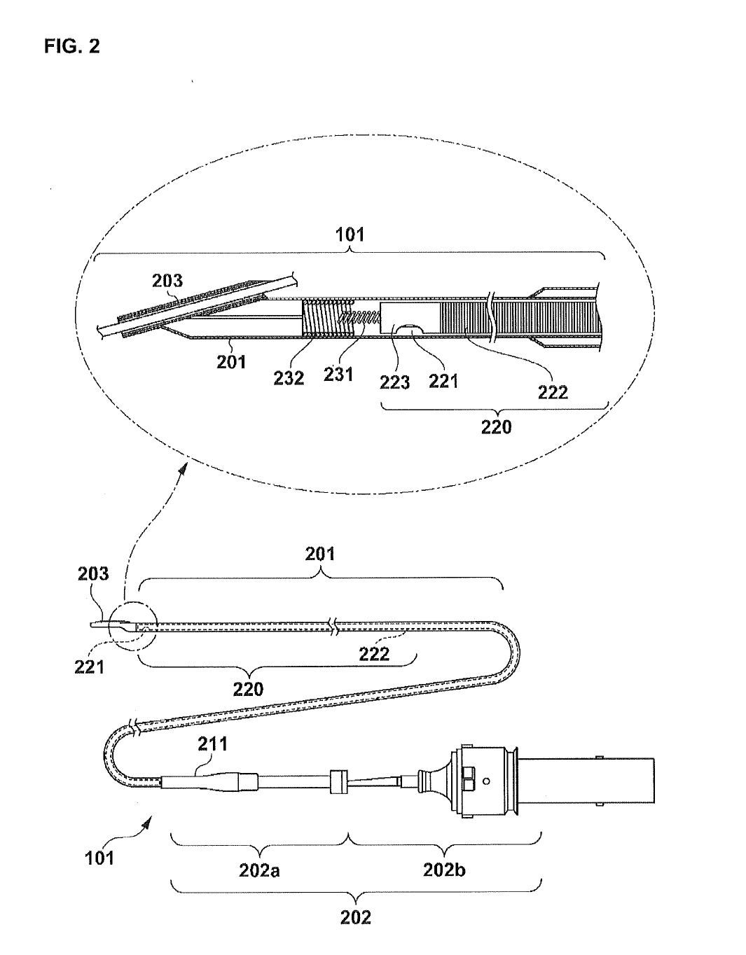

Next, an overall configuration of the probe unit 101 and a cross-sectional configuration of a distal end portion will be described with reference to FIG. 2. As illustrated in FIG. 2, the probe unit 101 is configured to include a long catheter sheath 201 to be inserted into the blood vessel and a connector unit 202 to be arranged on the front side of a user, and which can be operated by the user without being inserted into the blood vessel. The distal end of the catheter sheath 201 includes a tube 203 possessing a guide wire lumen configured to receive a guide wire. The catheter sheath 201 has a lumen, which is continuously formed from a connection portion with the guidewire lumen tube 203 to a connection portion with the connector unit 202.

An imaging core 220 which internally includes a transceiver 221 in which the ultrasound transceiver for transmitting and receiving the ultrasound waves and the optical transceiver for transmitting and receiving the light are arranged, and which includes a coil-shaped drive shaft 222 internally including an electrical signal cable and an optical fiber cable and transmitting rotary drive power for rotating the transceiver 221, which is inserted into the lumen of the catheter sheath 201 over substantially the entire length of the catheter sheath 201.

The connector unit 202 can include a sheath connector 202a configured to be integral with a proximal end of the catheter sheath 201, and a drive shaft connector 202b which is configured to rotatably fix the drive shaft 222 to the proximal end of the drive shaft 222.

An anti-kink protector 211 is disposed in a boundary section between the sheath connector 202a and the catheter sheath 201, which can help maintain predetermined rigidity, and can help prevent bending (kinking) caused by a rapid change in physical properties.

The proximal end of the drive shaft connector 202b is detachably attached to the scanner and pull-back unit 102.

Next, the cross-sectional configuration of the distal end portion of the probe unit 101 will be described. The imaging core 220 can include a housing 223 having the transceiver 221 in which the ultrasound transceiver for transmitting and receiving the ultrasound waves and the optical transceiver for transmitting and receiving the light are arranged, and including the drive shaft 222 for transmitting the rotary drive power for rotating the housing 223, which is inserted into the lumen of the catheter sheath 201 over substantially the entire length, thereby forming the probe unit 101.

The drive shaft 222 can cause the transceiver 221 to perform a rotary operation and an axial operation with respect to the catheter sheath 201. For example, the drive shaft 222 can be configured to have a multiplex and multilayer contact coil or the like formed of a metal wire having a property, which can be flexible and can transmit rotation well such as a stainless steel wire or the like. Then, an electric signal cable and an optical fiber cable (optical fiber cable in a single mode) can be arranged inside the drive shaft 222.

The housing 223 has a shape in which a short cylindrical metal pipe partially has a cutout portion, and is formed by being cut out from a metallic ingot, or is molded by means of metal powder injection molding (MIM), for example. In addition, an elastic member 231 having a short coil shape can be disposed on the distal end side of the housing 223.

The elastic member 231 is obtained by forming a stainless steel wire into a coil shape. The elastic member 231 is arranged on the distal end side, thereby help preventing the imaging core 220 from being caught on the inside of the catheter sheath 201 when the imaging core 220 is moved forward and rearward.

The reference numeral 232 represents a reinforcement coil, which is disposed in order to help prevent rapid bending of the distal end portion of the catheter sheath 201.

The guidewire lumen tube 203 has a guidewire lumen into which a guidewire can be inserted. The guidewire lumen tube 203 is used in receiving the guidewire inserted into the blood vessel in advance and allowing the guidewire to guide the catheter sheath 201 to a lesion.

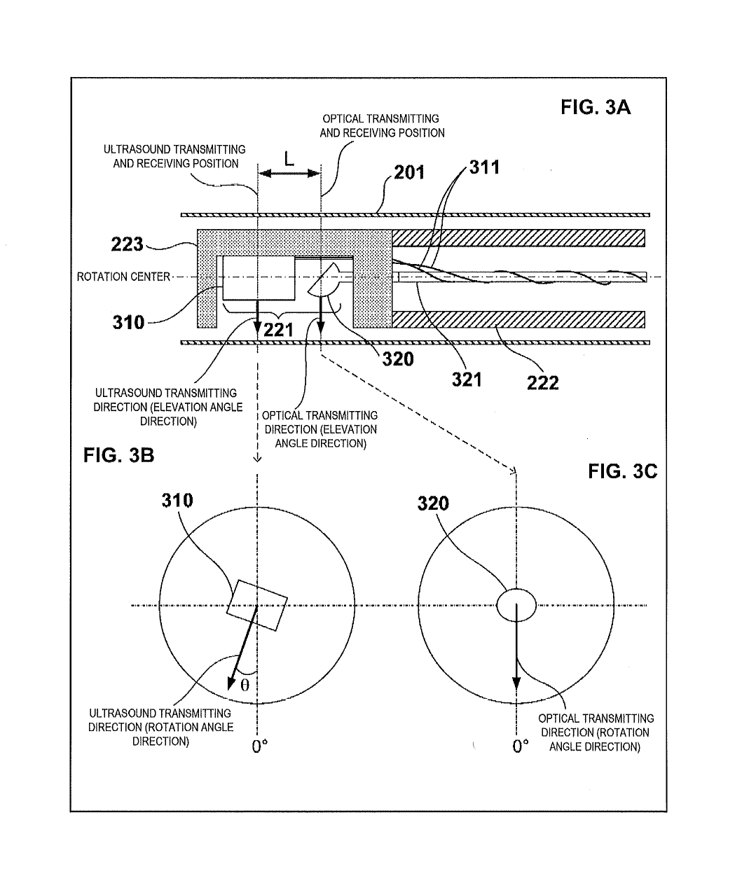

Next, a cross-sectional configuration of the imaging core 220 and an arrangement for the ultrasound transceiver and the optical transceiver will be described. FIGS. 3A-3C are diagrams illustrating the cross-sectional configuration of the imaging core, the arrangement for the ultrasound transceiver, and the optical transceiver, respectively.

As illustrated in FIG. 3A, the transceiver 221 arranged inside the housing 223 can include an ultrasound transceiver 310 and an optical transceiver 320. The ultrasound transceiver 310 and the optical transceiver 320 can be respectively arranged along the axial direction on the rotation center axis (on the one-dot chain line in FIG. 3A) of the drive shaft 222.

In accordance with an exemplary embodiment, the ultrasound transceiver 310 can be arranged on the distal end side of the probe unit 101, and the optical transceiver 320 can be arranged on the proximal end side of the probe unit 101.

In addition, the ultrasound transceiver 310 and the optical transceiver 320 are attached inside the housing 223 so that an ultrasound transmitting direction (elevation angle direction) of the ultrasound transceiver 310 and a light transmitting direction (elevation angle direction) of the optical transceiver 320 are respectively, for example, approximately 90° with respect to the axial direction of the drive shaft 222. In accordance with an exemplary embodiment, the ultrasound transceiver 310 can be attached to the optical transceiver 320 by causing each transmitting direction to be slightly deviated from 90° so as not to receive the reflection on a surface inside the lumen of the catheter sheath 201.

An electric signal cable 311 connected to the ultrasound transceiver 310 and an optical fiber cable 321 connected to the optical transceiver 320 are arranged inside the drive shaft 222. The electric signal cable 311 can be wound around the optical fiber cable 321 in a spiral shape.

FIG. 3B is a cross-sectional view taken along a plane substantially orthogonal to the rotation center axis at an ultrasound transmitting and receiving position. As illustrated in FIG. 3B, when a downward direction from the paper surface is zero degrees, the ultrasound transmitting and receiving direction (rotation angle direction (also referred to as an azimuth angle direction)) of the ultrasound transceiver 310 is θ degrees.

FIG. 3C is a cross-sectional view taken along a plane substantially orthogonal to the rotation center axis at an optical transmitting and receiving position. As illustrated in FIG. 3C, when the downward direction from the paper surface is zero degrees, the light transmitting and receiving direction (rotation angle direction) of the optical transceiver 320 is zero degrees. That is, the ultrasound transceiver 310 and the optical transceiver 320 are arranged so that the ultrasound transmitting and receiving direction (rotation angle direction) of the ultrasound transceiver 310 and the light transmitting and receiving direction (rotation angle direction) of the optical transceiver 320 are deviated from each other by θ degrees.

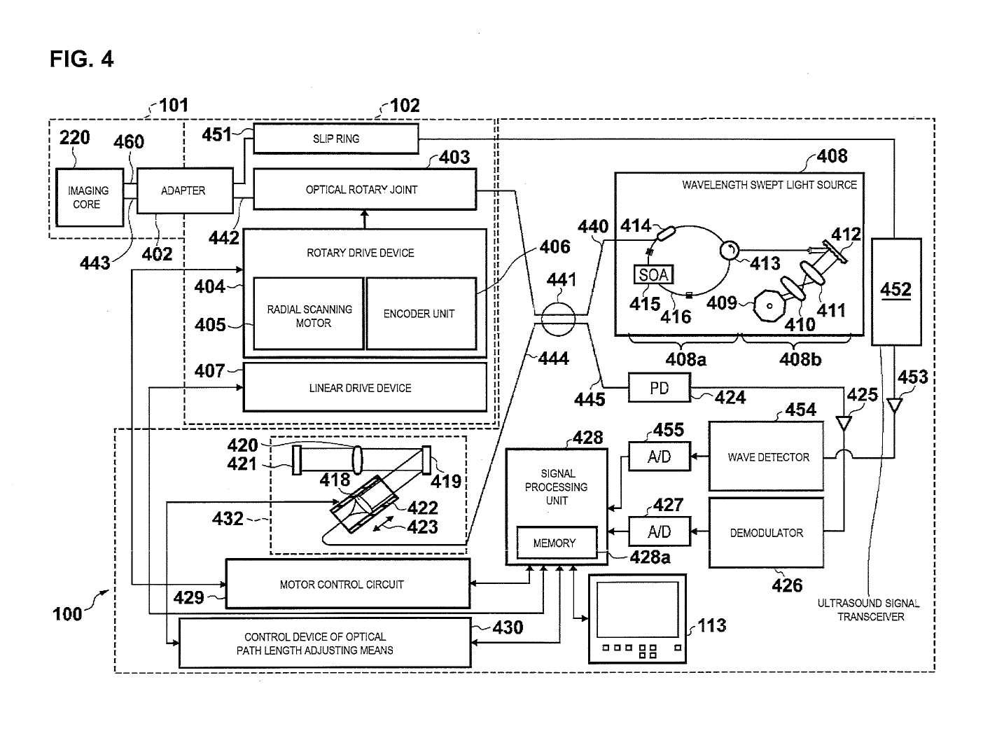

Next, a functional configuration of the imaging apparatus for diagnosis 100 will be described. FIG. 4 is a diagram illustrating the functional configuration of the imaging apparatus for diagnosis 100 which includes an IVUS function and an OCT function (here, a swept source OCT as an example) in combination. An imaging apparatus for diagnosis including the IVUS function and other OCT functions in combination also has the same functional configuration. Therefore, description thereof will be omitted herein.

The imaging core 220 can include the ultrasound transceiver 310 inside the distal end of the image core 220. The ultrasound transceiver 310 can transmit ultrasound waves to biological tissues based on pulse waves transmitted by an ultrasound signal transceiver 452, receives reflected waves (echoes) of the ultrasonic waves, and transmits the reflected waves to the ultrasound signal transceiver 452 as an ultrasound signal via an adapter 402 and a slip ring 451.

In the scanner and pull-back unit 102, a rotary drive portion side of the slip ring 451 is rotatably driven by a radial scanning motor 405 of a rotary drive device 404. In addition, a rotation angle of the radial scanning motor 405 is detected by an encoder unit 406. Furthermore, the scanning and pull-back unit 102 can include a linear drive device 407, and can regulate the axial operation of the imaging core 220 based on a signal from a signal processing unit 428.

The ultrasound signal transceiver 452 can include a transmitting wave circuit and a receiving wave circuit (not illustrated). The transmitting wave circuit transmits the pulse waves to the ultrasound transceiver 310 inside the imaging core 220 based on a control signal transmitted from the signal processing unit 428.

In addition, the receiving wave circuit receives an ultrasound signal from the ultrasound transceiver 310 inside the imaging core 220. The received ultrasound signal can be amplified by an amplifier 453, and then can be input to and detected by a wave detector 454.

Furthermore, an A/D converter 455 can generate digital data (ultrasound data) of one line by sampling the ultrasound signal output from the wave detector 454 at, for example, 30.6 MHz by an amount of 200 points. Although 30.6 MHz is used here, this is calculated on the assumption that the sampling of 200 points is performed for a depth of 5 mm when sound velocity is set to 1530 m/sec. Therefore, the sampling frequency is not particularly limited thereto.

The ultrasound data in units of lines which is generated by the A/D converter 455 is input to the signal processing unit 428. The signal processing unit 428 can convert the ultrasound data into a gray scale, thereby generating an ultrasound cross-sectional image (hereinafter, referred to as an IVUS cross-sectional image) at each position inside a blood vessel and outputting the ultrasound cross-sectional image to an LCD monitor 113 at a predetermined frame rate.

The signal processing unit 428 is connected to a motor control circuit 429, and receives a video synchronization signal of the motor control circuit 429. The signal processing unit 428 can generate the ultrasound cross-sectional image in synchronization with the received video synchronization signal.

In addition, the video synchronization signal of the motor control circuit 429 can also be transmitted to the rotary drive device 404, and the rotary drive device 404 can output a drive signal synchronized with the video synchronization signal.

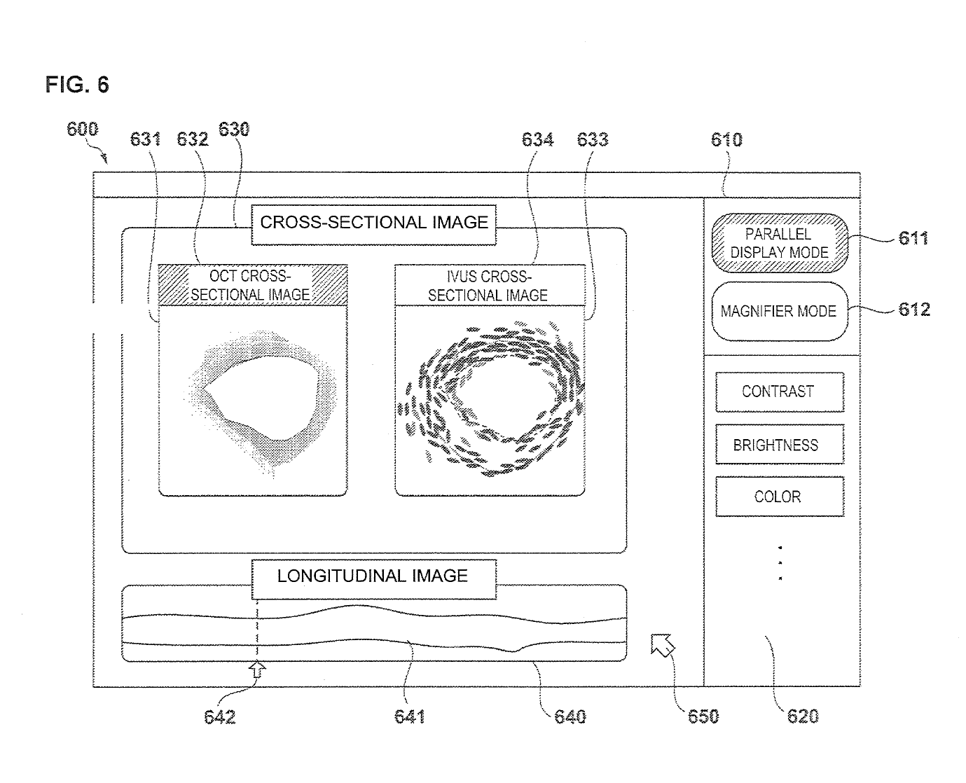

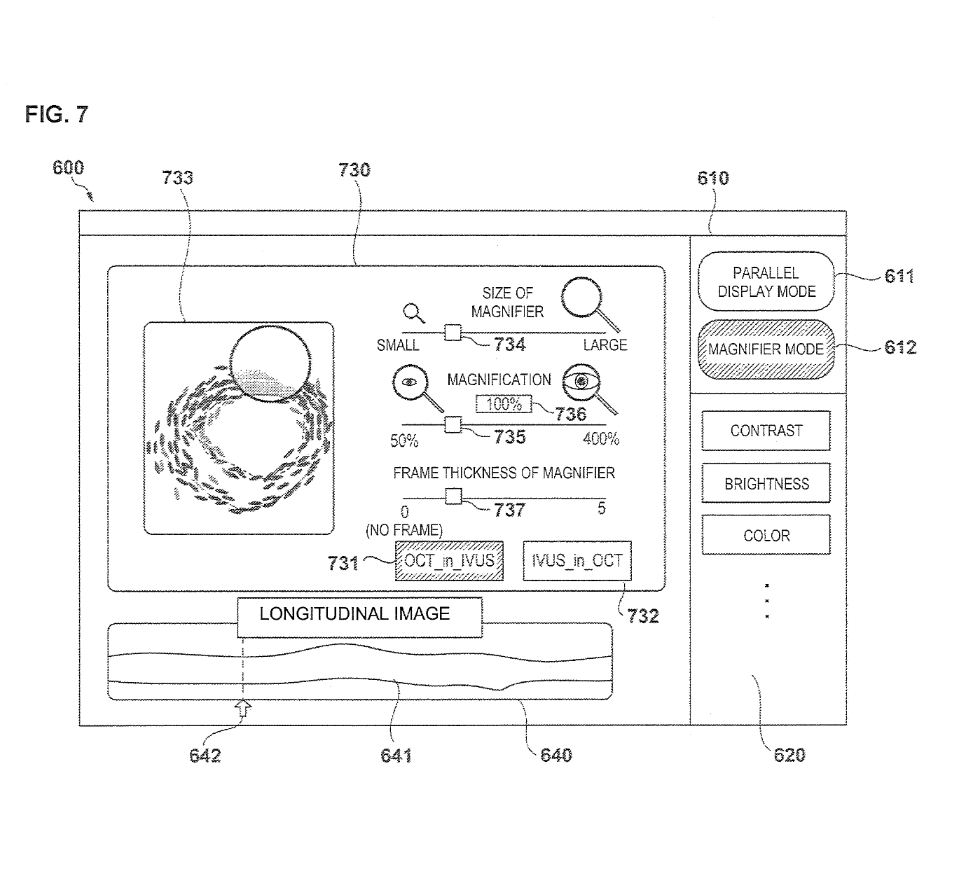

The above-described processing in the signal processing unit 428 and image processing relating to a user interface in the imaging apparatus for diagnosis 100 (to be described later with reference to FIGS. 6 and 7) can be realized in such a way that a predetermined program can cause a computer to execute the processing in the signal processing unit 428.

Next, a functional configuration of swept source OCT will be described with reference to the same drawings. The reference numeral 408 represents a wavelength swept light source (swept laser), and is one type of an extended-cavity laser which can include an optical fiber 416 which is coupled to a semiconductor optical amplifier (SOA) 415 in a ring shape and a polygon scanning filter (408b).

Light output from the SOA 415 proceeds to the optical fiber 416, and enters the polygon scanning filter 408b. In accordance with an exemplary embodiment, the light whose wavelength is selected here can be amplified by the SOA 415, and can be finally output from a coupler 414.

In accordance with an exemplary embodiment, the polygon scanning filter 408b selects the wavelength in combination with a diffraction grating 412 for diffracting the light and a polygon mirror 409. For example, the light diffracted by the diffraction grating 412 can be concentrated on a surface of the polygon mirror 409 by two lenses (410 and 411). In this manner, only the light having a wavelength orthogonal to the polygon mirror 409 returns through the same optical path, and is output from the polygon scanning filter 408b. In accordance with an exemplary embodiment, time sweeping of the wavelength can be performed by rotating the polygon mirror 409.

For example, a 48-sided mirror can be used for the polygon mirror 409 whose rotation speed is approximately 50000 rpm. A wavelength sweeping system in which the polygon mirror 409 and the diffraction grating 412 can be combined with each other, which can enable high speed and high output wavelength sweeping.

The light of a wavelength swept light source 408 which is output from the coupler 414 is incident on one end of a first single mode fiber 440, and is transmitted to the distal end side. The first single mode fiber 440 can be optically coupled to a second single mode fiber 445 and a third single mode fiber 444 in an optical coupler 441 located in the middle therebetween.

On the further distal end side than the optical coupler 441 of the first single mode fiber 440, an optical rotary joint (optical coupling unit) 403 which transmits the light by coupling a non-rotating part (fixed portion) and a rotating part (rotary drive unit) to each other can be disposed inside the rotary drive device 404.

Furthermore, a fifth single mode fiber 443 of the probe unit 101 can be detachably connected via the adapter 402 to the distal end side of a fourth single mode fiber 442 inside the optical rotary joint (optical coupling unit) 403. In this manner, the light from the wavelength swept light source 408 can be transmitted to the fifth single mode fiber 443 which is inserted into the imaging core 220 and can be rotatably driven.

The transmitted light is emitted from the optical transceiver 320 of the imaging core 220 to the biological tissues inside the blood vessel while a rotary operation and an axial operation can be performed. Then, the reflected light scattered on a surface or inside the biological tissues can be partially captured by the optical transceiver 320 of the imaging core 220, and returns to the first single mode fiber 440 side through a rearward optical path. Furthermore, the light can be partially transferred to the second single mode fiber 445 side by the optical coupler 441, and can be emitted from one end of the second single mode fiber 445. Thereafter, an optical detector, for example, a photodiode 424 can receive the light.

The rotary drive unit side of the optical rotary joint 403 can be rotatably driven by the radial scanning motor 405 of the rotary drive device 404.

In accordance with an exemplary embodiment, an optical path length variable mechanism 432 for finely adjusting an optical path length of reference light can be disposed in the distal end opposite to the optical coupler 441 of the third single mode fiber 444.

In order for variations in the length of an individual probe unit 101 to be absorbed when the probe unit 101 is replaced and newly used, this optical path length variable mechanism 432 can include optical path length changing means for changing an optical path length corresponding to the variations in the length.

The third single mode fiber 444 and a collimating lens 418 can be disposed on a one-axis stage 422 which is movable in an optical axis direction thereof as illustrated by an arrow 423, thereby forming the optical path length changing means.

In accordance with an exemplary embodiment, the one-axis stage 422 functions as the optical path length changing means having a variable enough range of the optical path length to absorb the variations in the optical path length of the probe unit 101 when the probe unit 101 is replaced. Furthermore, the one-axis stage 422 also can include a function as adjusting means for adjusting an offset. For example, even when the distal end of the probe unit 101 is not in close contact with the surface of the biological tissues, the one-axis stage can finely change the optical path length. In this manner, the optical path length can be set in a state of interfering with the reflected light from the surface position of the biological tissues.

In accordance with an exemplary embodiment, the optical path length can be finely adjusted by the one-axis stage 422. The light reflected on a mirror 421 via a grating 419 and a lens 420 can be mixed with the light obtained from the first single mode fiber 440 side by the optical coupler 441 disposed in the middle of the third single mode fiber 444, and then can be received by the photodiode 424.

Interference light received by the photodiode 424 in this way can be photoelectrically converted, and can be input to a demodulator 426 after being amplified by the amplifier 425. The demodulator 426 can perform demodulation processing for extracting only a signal portion of the interference light, and an output therefrom can be input to the A/D converter 427 as an interference light signal.

The A/D converter 427 can perform sampling on the interference light signal, for example, at 180 MHz by an amount of 2048 points, and can generate digital data (interference light data) of one line. In accordance with an exemplary embodiment, the reason for setting the sampling frequency to 180 MHz can be based on the assumption that approximately, for example, 90% of wavelength sweeping cycles (12.5 μsec) can be extracted as the digital data of 2048 points, when a repetition frequency of the wavelength sweeping is set to 80 kHz. However, the sampling frequency is not particularly limited thereto.

In accordance with an exemplary embodiment, the interference light data in the unit of lines which is generated by the A/D converter 427 can be input into the signal processing unit 428. The signal processing unit 428 can generate data in a depth direction (line data) by performing frequency resolution on the interference light data using the fast Fourier transform (FFT), and the data can be subjected to coordinate transformation. In this manner, an optical cross-sectional image (hereinafter, referred to as an OCT cross-sectional image) can be constructed at each intravascular position, and can be output to the LCD monitor 113 at a predetermined frame rate.

In accordance with an exemplary embodiment, the signal processing unit 428 can be further connected to a control device of optical path length adjusting means 430. The signal processing unit 428 can control a position of the one-axis stage 422 via the control device of optical path length adjusting means 430.

The processing in the signal processing unit 428 can also be realized in such a way that a predetermined program can cause a computer to execute the processing.

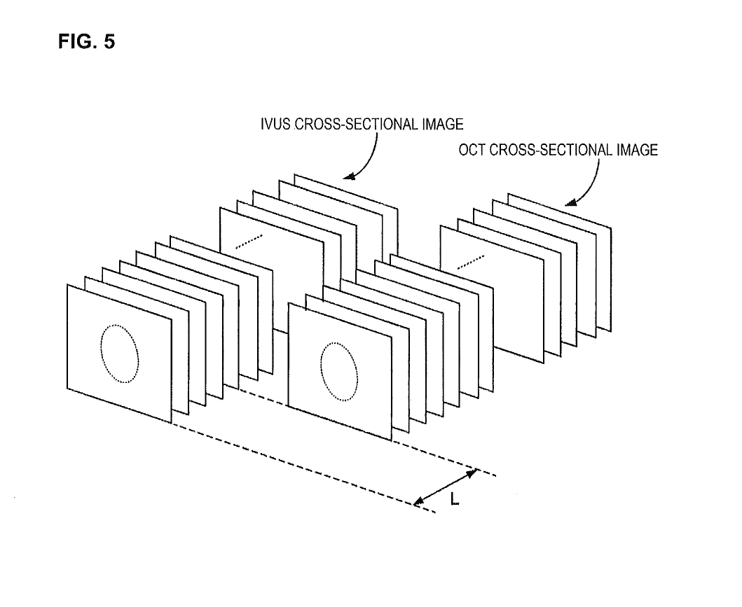

In the above-described configuration, if a user inputs an instruction to start scanning by operating the operation control device 103, the signal processing unit 428 controls the scanner and pull-back unit 102. In this manner, the imaging core 220 is rotated, and the scanner and pull-back unit 102 is moved in the longitudinal direction of the blood vessel by pulling the imaging core 220 at a predetermined speed. As a result, as described above, the A/D converters 427 and 455 output the digital ultrasound data and the interference light data. Accordingly, the signal processing unit 428 can build the ultrasound cross-sectional image and the optical cross-sectional image at each position in the data along the movement direction of the imaging core 220, and can store both of these in the memory 428a included in the signal processing unit 428. In this case, scales of the ultrasound cross-sectional image and the optical cross-sectional image can be aligned with each other, and furthermore, the central position of the respective cross-sectional images can be aligned with a rotation axis at the time of scanning. FIG. 5 illustrates an example of the ultrasound cross-sectional image and the optical cross-sectional image both of which are stored in the memory 428a included in the signal processing unit 428. As described above, the emitting directions of the ultrasound transceiver 310 and the optical transceiver 320 can be deviated by θ as illustrated in FIG. 3B. Accordingly, when the cross-sectional images are configured, one emitting direction can be deviated by θ, thereby aligning orientations of the two types of the cross-sectional image with each other. As illustrated in FIG. 3A, the ultrasound transceiver 310 and the optical transceiver 320 can be deviated by L from the movement direction of the imaging core 220 using a pull-back operation. Accordingly, in order to obtain the ultrasound cross-sectional image and the optical cross-sectional image at the same position in the blood vessel, the cross-sectional image to be rebuilt is deviated by L as illustrated in FIG. 5. For example, in order to obtain an ultrasound cross-sectional image at a position corresponding to a certain optical cross-sectional image, the ultrasound cross-sectional image can be acquired at a position deviated by L.

In accordance with an exemplary embodiment, the above-described θ and L may be set in advance by operating the operation control device 103 when the scanning starts.

Next, a user interface displayed on the LCD monitor 113 will be described. In the following description, description will be made on the assumption that the scanning inside the blood vessel of a patient has already been completed and a process for generating the cross-sectional image at each position as illustrated in FIG. 5 has already been completed. In addition, various indications described below are input by using the operation panel 112 or the mouse 114.

FIG. 6 illustrates a user interface 600 in a parallel display mode after the scanning, which is displayed on the LCD monitor 113. The illustrated user interface 600 can be configured to include four display regions 610, 620, 630, and 640. In addition, a cursor 650 to be displayed in conjunction with the mouse 114 can be displayed.

In accordance with an exemplary embodiment, the display region 610 has a parallel display mode button 611 for indicating a display mode and a magnifier mode button 612. An operation of clicking the button of the mouse 114 by operating the mouse 114 and moving the cursor 650 onto the button 611 can be referred to as “clicking of the button 611”.

As described above, FIG. 6 illustrates the parallel display mode for displaying the IVUS cross-sectional image and the OCT cross-sectional image side by side. Accordingly, as illustrated, the parallel display mode button 611 can be displayed by being highlighted. The user interface displayed when the magnifier mode button 612 is clicked will be described later.

The region 620 has various image processing buttons for the selected cross-sectional image of the displayed IVUS cross-sectional image or the displayed OCT cross-sectional image. For example, if a contrast button illustrated in the drawing is clicked, contrast-related setting can be changed for the cross-sectional image selected at that time. Types of the image processing are not limited. However, in order to enable multiple image processing buttons to be displayed, a scroll bar may be disposed, or the respective image processing buttons may be displayed by using a tab display format.

The region 630 can include an OCT cross-sectional image display region 631 and an IVUS cross-sectional image display region 633. In addition, in order to indicate which types of the image can be respectively displayed, image type identifying labels 632 and 634 can be added to the upper portion of the respective cross-sectional images. If a user clicks the inside of the OCT cross-sectional image display region 631 or the IVUS cross-sectional image display region 633, the cross-sectional image on which the cursor is located when clicked is selected as a target for the various image processing. For example, as shown in FIG. 6, since the label 632 is displayed by being highlighted, it is identified that the OCT cross-sectional image is selected. If the user clicks the inside of the IVUS cross-sectional image display region 633, the IVUS cross-sectional image can be selected as a target for the image processing. Accordingly, the label 634 is displayed by being highlighted.

A cross-sectional image 641 in the longitudinal direction of the blood vessel which is generated based on multiple IVUS cross-sectional images (alternatively, based on multiple OCT cross-sectional images) is displayed in the region 640. In addition, a cross-sectional image in the longitudinal direction of the blood vessel, which is generated, based on the IVUS cross-sectional image and an OFDI cross-sectional image may be concurrently displayed in the region 640. In accordance with an exemplary embodiment, a marker 642 inside the display region can indicate a position of the cross-sectional images displayed in the regions 610 and 620. The position of the marker 642 can be changed by operating the mouse 114. For example, the cursor 650 can be moved onto the marker 642, and the mouse 114 can be moved while a mouse button is pressed down (in general, called a drag operation). In this manner, the marker 642 moves along the horizontal direction. Based on a position of the moving marker 642, the signal processing unit 428 reads out the OCT cross-sectional image and the IVUS cross-sectional image at the position from the memory 428a, and performs a process for displaying the images in the display regions 631 and 633.

Hitherto, the user interface in FIG. 6 has been described. When an operator examines internal conditions of a patient's blood vessel, the operator operates the mouse 114, and freely moves the marker 642. Each time, the operator performs diagnosis on the patient's blood vessel while viewing the IVUS cross-sectional image and the OCT cross-sectional image, which can be displayed in the regions 631 and 633.

Whereas the OCT cross-sectional image can enable the operator to obtain a high resolution image for relatively shallow tissues, the OCT cross-sectional image is not suitably used in obtaining an image for deep tissues. For example, the IVUS cross-sectional image can have inferior resolution to the OCT cross-sectional image, but can enable the operator to obtain an image for the relatively deep tissues. For example, it can be considered that the OCT cross-sectional image and the IVUS cross-sectional image are in a relationship to compensate for each other. Therefore, if these two images can be concurrently checked without changing the viewpoint, this method can advantageously be used in diagnosis. In accordance with an exemplary embodiment, a method is disclosed where two images can be displayed by being synthesized to generate one synthesized image. However, if these two images are displayed by being synthesized at a ratio of, for example, 50:50, original contrast belonging to each image becomes half of each original image, thereby hindering the diagnosis.

Therefore, in accordance with an exemplary embodiment, a configuration can be adopted in which one of the OCT cross-sectional image and the IVUS cross-sectional image can be displayed as a reference image and the other cross-sectional image can be visible through a virtual magnifier. Furthermore, a position of the magnifier can be configured to be freely changeable by a user's operation. This display mode is a magnifier mode. The process proceeds to this mode by clicking the magnifier mode button 612 in the region 610.

FIG. 7 illustrates the user interface 600 during the magnifier mode according to an exemplary embodiment. In the illustration shown in FIG. 7, since the regions 610, 620, and 640 are the same, as those in FIG. 6, description thereof will be omitted. However, since the user interface in the magnifier display mode is illustrated, the magnifier mode button 612 can be displayed by being highlighted in the region 610.

In FIG. 7, a region 730 is a region displayed in place of the region 630 in FIG. 6, and is configured to have buttons 731 and 732 for a user's instruction, an image display region 733 for displaying a reference image, a slider 734 for indicating a size of the magnifier, a slider 735 for indicating magnification M of the magnifier, a region 736 (“100%” is displayed in a case of default) for indicating magnification M (percentage) indicated by the slider 735, and a slider 737 for indicating thickness of a circular frame showing the magnifier.

The button 731 is a button for causing the process to proceed to a mode where the IVUS cross-sectional image is set to a reference image and an image viewable through the magnifier is set to the OCT cross-sectional image. In the illustration, the button 731 indicates “OCT_in_IVUS”, which represents that the OCT cross-sectional image is displayed inside the IVUS cross-sectional image. When the process proceeds to the magnifier mode, the button 731 is in a state selected by default.

The button 732 is a button for causing the process to proceed to a mode where the OCT cross-sectional image is set to the reference image and an image viewable through the magnifier is set to the IVUS cross-sectional image. In the illustration, the button 732 indicates “IVUS_in_OCT”, which represents that the IVUS cross-sectional image is displayed inside the OCT cross-sectional image.

For example, in the magnifier mode, two modes of an OCT_in_IVUS mode and an IVUS_in_OCT mode are present as a lower level mode.

In accordance with an exemplary embodiment, any one of the OCT_in_IVUS mode and the IVUS_in_OCT mode may be selected. Accordingly, any one of the two buttons 731 and 732 may be eliminated. For example, a configuration may be adopted in which one button is turned on or turned off so as to switch two modes to each other.

In accordance with the mode selected by any one of the buttons 731 and 732, either the IVUS cross-sectional image or the OCT cross-sectional image is displayed as the reference image in the image display region 733. Then, a circular frame showing the magnifier is disposed in the region 734, and the other cross-sectional image different from the reference image can be displayed inside the circular frame. In a case of FIG. 6, the button 731 indicating OCT_in_IVUS is displayed by being highlighted. Accordingly, the IVUS cross-sectional image is displayed in the image display region 733 as the reference image, and the OCT cross-sectional image is displayed in the circular frame. When the process proceeds to the IVUS_in_OCT mode by clicking the button 732, the reference image becomes the OCT cross-sectional image, and the IVUS cross-sectional image is displayed inside the circular frame.

The thickness of the circular frame can be freely changed by laterally moving the slider 737. In the embodiment, the thickness of the circular frame is set to have six stages from zero to five, but the setting is an example. When the thickness of the circular frame is set to zero, the circular frame is in a non-displayed state. For example, the slider 737 can also serve to cause the circular frame to be switched between display and non-display. Even when the circular frame is not displayed, the circular frame used in cutting out or writing an image is present on operation (to be described later). In addition, description can be made so that a color of the circular frame is preset, but a configuration may be adopted in which the color of the circular frame can be changed freely.

Hereinafter, referring to FIG. 7, a case will be further described where the OCT_in_IVUS mode is indicated in the magnifier mode.

In accordance with an exemplary embodiment, a size of the circular frame inside the image display region 733 is a size depending on a position of the slider 734. In addition, magnification of the OCT cross-sectional image displayed inside the circular frame can depend on a position of the slider 735.

The position of the circular frame can be changed by causing the cursor 650 moving in conjunction with the mouse 114 to move inside the image display region 733 for the time being. For example, as long as a position indicated by the mouse 114 is located inside the image display region 733, instead of the cursor 650, a user comes to operate a position of the circular frame in turn. The cursor 650 is not displayed in the user interface of FIG. 7. Accordingly, the user interface shows that the position indicated by the user is located inside the image display region 733.

Since the configuration is adopted as described above, if the user moves the circular frame inside the image display region 733 by operating the mouse 114, the OCT cross-sectional image corresponding to the center position of the circular frame can be partially subjected to magnification processing according to the magnification M at that time, and can be displayed inside the circular frame. As a result, the user can observe an image located at a user's interest position inside the IVUS cross-sectional image as if the user views the image as the OCT cross-sectional image through the magnifier. Moreover, it means that the magnification M can be freely set by the user.

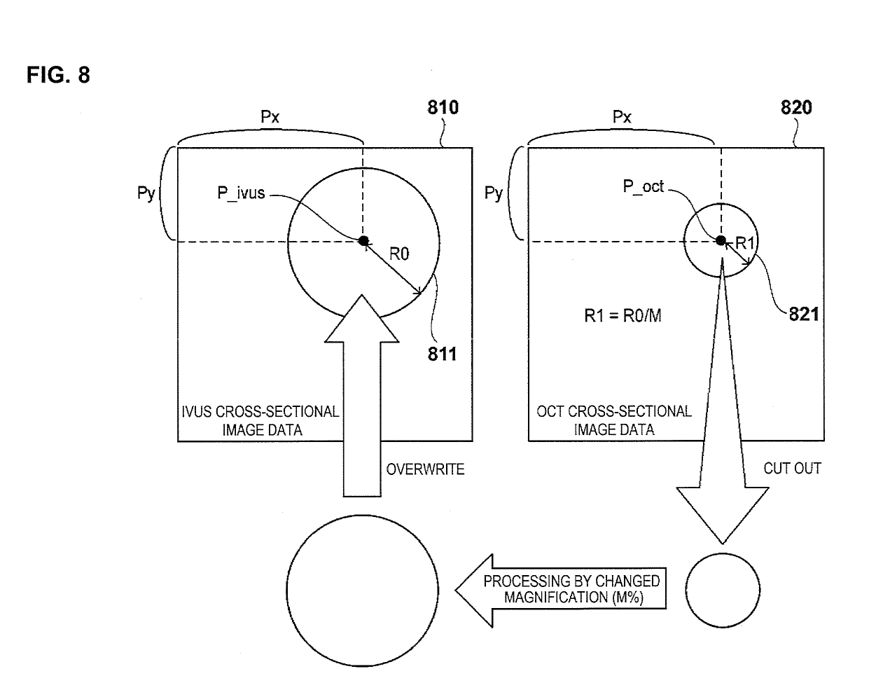

A process of the signal processing unit 428 in the above-described OCT_in_IVUS mode will be further described in detail with reference to FIG. 8.

From the memory 428a, the signal processing unit 428 reads IVUS cross-sectional image data 810 and OCT cross-sectional image 820 which are specified at the position of the marker 642. Here, scales of the IVUS cross-sectional image data 810 and the OCT cross-sectional image 820 are the same as each other, and also coincide with the scale of the image display region 733.

In the OCT_in_IVUS mode, since the IVUS cross-sectional image can be displayed as the reference image in the image display region 733 illustrated in FIG. 7, it may be considered that the circular frame illustrated in FIG. 7 is located on the IVUS cross-sectional image data 810. Therefore, it is considered that the circular frame in FIG. 7 is a circular frame 811 in FIG. 8.

In accordance with an exemplary embodiment, the following procedures may be followed in order to partially magnify and display the OCT cross-sectional image data 820 in the circular frame 811.

(1) From the memory 428a, read the OCT cross-sectional image data 820 and the IVUS cross-sectional image data 810 which correspond to the position of the marker 642.

(2) Cut out a partial image inside a circular region 821 which is a target for magnified display inside the OCT cross-sectional image data 820.

(3) Magnify the cut-out partial image inside the circular region 821 in accordance with the magnification M at that time.

(4) Overwrite the image obtained by the magnification processing inside the circular frame 811 of the IVUS cross-sectional image data 810, and display the result.

A center point P_oct of the circular region 821 in the above-described Step (2) is the same as coordinates of a center point P_ivus of the circular frame 811. A radius R1 of the circular region 821 and a radius RO of the circular frame 811 are different from each other. A case has been already described where the radius RO of the circular frame 811 is determined depending on the position of the slider 734. For example, in accordance with an exemplary embodiment, the radius R1 of the circular region 821 can be expressed by the magnification M and the radius RO of the circular frame 811 as in the following equation.

R1=R0/M

That is, when the magnification is 100%, the result shows R1=R0, and when the magnification is 200%, the result shows R1=R0/2.

Therefore, the signal processing unit 428 cuts out the partial image inside the circular region having the radius R1 (=R0/M) which is centered on the position P-oct indicated by the mouse 114, from the OCT cross-sectional image data 820.

In Step (3), the signal processing unit 428 performs the magnification processing on the cut-out partial image based on the magnification M. Through this magnification processing, a circular image having the radius R0 can be generated. Various methods of the magnification processing are known, but linear interpolation processing is applied here.

In Step (4), the signal processing unit 428 overwrites the generated magnified image inside the circular frame 811 in the IVUS cross-sectional image data 810. Then, the signal processing unit 428 displays the IVUS cross-sectional image data 810 (partially rewritten to the OCT cross-sectional image data) after the overwriting process.

In accordance with an exemplary embodiment, the signal processing unit 428 repeatedly performs the above-described processing as long as a user operates the mouse 114 to change the indicating position and the changed indicating position is located inside the image display region 733.

As a result of the above-described processing, the position of the circular frame (magnifier) in FIG. 7 can be freely changed according to the user's intention. The IVUS cross-sectional image can be displayed outside the circular frame indicating the magnifier within the image display region 733, and a partial image of the OCT cross-sectional image is displayed inside the circular frame. Here, the position of the circular frame can be freely moved by a user. Accordingly, the user can view the OCT cross-sectional image as if the OCT cross-sectional image is displayed in a range of the IVUS cross-sectional image, which is “peeped” through the magnifier. Furthermore, if the user wants to check the IVUS cross-sectional image hidden in the OCT cross-sectional image inside the circular frame, the hidden IVUS cross-sectional image may be sufficiently displayed by simply shifting the current position of the circular frame. That is, for the user, as a matter of course, the user's desired site can be checked by using the OCT cross-sectional image without changing the user's viewpoint. Moreover, the user's desired site can be checked by using the IVUS cross-sectional image.

The above-described processing is performed in the OCT_in_IVUS mode. However, processing in the IVUS_in_OCT mode is performed by only changing the term of the “OCT cross-sectional image” in the above-described processing to the term of the “IVUS cross-sectional image”, and by only changing the term of the “IVUS cross-sectional image” in the above-described processing to the term of the “OCT cross-sectional image”. Accordingly, description thereof will be omitted.

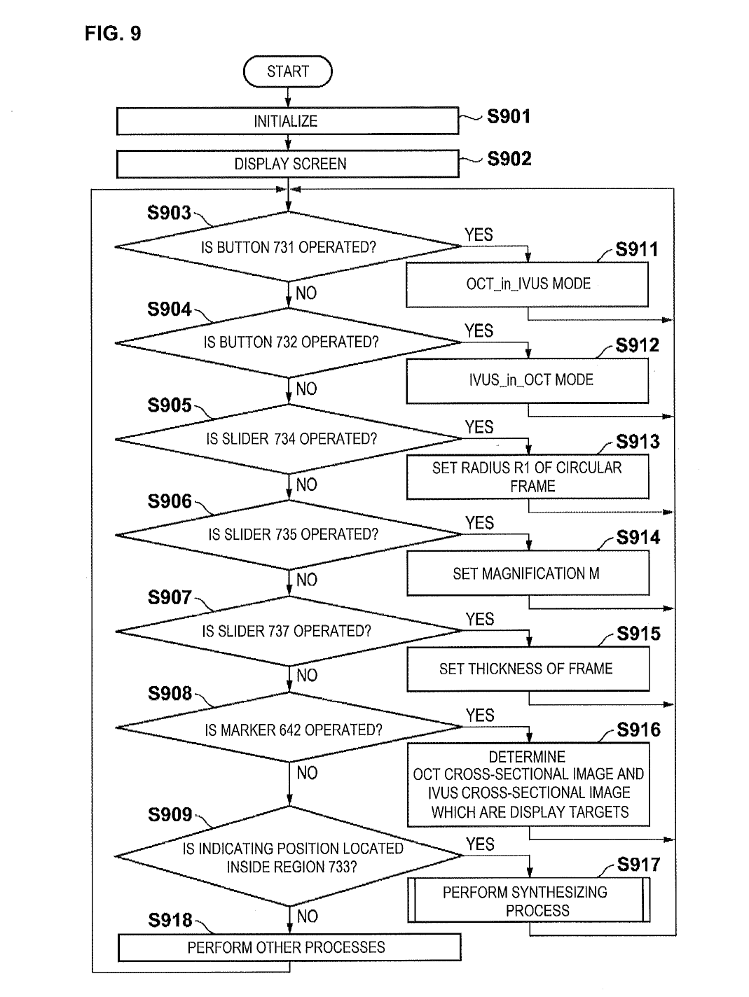

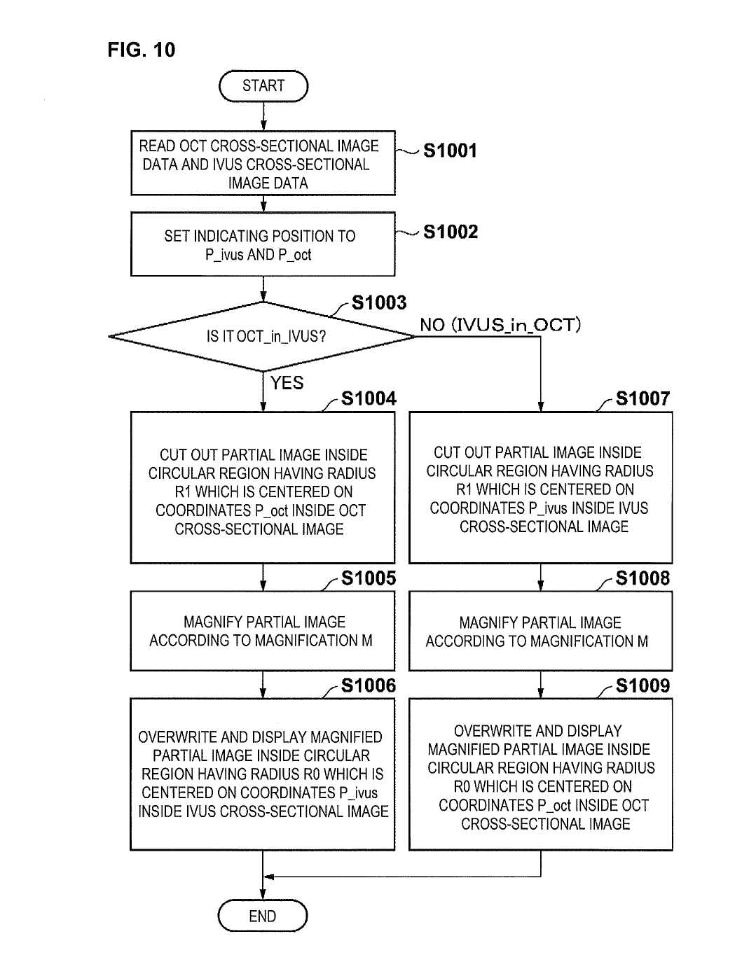

Characteristics in the embodiment show processing according to the user interface in FIG. 7. Therefore, hereinafter, processing procedures of the signal processing unit 428 in the display of the user interface in FIG. 7 will be described with reference to a flowchart in FIGS. 9 and 10. A program according to the processing procedures with reference to the illustrated flowchart can be stored in a hard disk device or the like.

First, in Step S901, an initializing process is performed. The initializing process can include a process for setting the OCT_in_IVUS mode as a default mode, a process for setting the thickness and the radius R0 of the circular frame, and for setting the magnification M to be an initial value (in the embodiment, 100%), and a process for setting an initial position of the marker 642, for example.

Next, in Step S902, based on the result of the initialized process, a screen of the user interface of FIG. 7 is displayed on the LCD monitor 113.

Thereafter, in Steps S903 to S909, a user's operation target can be determined on the user interface of FIG. 7.

When the button 731 is clicked, the display mode is set to the OCT_in_IVUS mode in Step S911. At this time, the IVUS cross-sectional image serving as the reference image can be selected as a target for various image processing.

In addition, when the button 732 is determined to be clicked, the display mode is set to the IVUS_in_OCT mode in Step S912. At this time, the OCT cross-sectional image serving as the reference image is selected as a target for various image processing.

When the slider 734 is determined to be operated, the radius R1 of the circular frame is updated according to the position of the slider 734 in Step S913. In addition, when the slider 735 is determined to be operated, the magnification M is updated according to the position of the slider 735 in Step S914.

When a check box 737 is determined to be operated, the process proceeds to Step S915 so as to determine the thickness of the circular frame. A case has been already described where the circular frame is not displayed when the thickness is zero.

When the marker 642 is determined to be operated, the OCT cross-sectional image data and the IVUS cross-sectional image data which are display targets are determined according to the positions in Step S916.

In addition, when the user's indicating position (cursor 650) is determined to be located inside the image display region 733, the process proceeds to Step S917 so as to perform a synthesizing process (to be described later).

Then, when others except for the above-described one are determined to be operated, a corresponding process is performed in Step S918. The process in Step S918 is a process according to the various buttons inside the regions 610 and 630. For example, when the contrast button is clicked, the process proceeds to an adjustment process of contrast for the selected cross-sectional image. In addition, when the parallel display mode button 611 is clicked, the user interface of FIG. 7 is switched over to the user interface of FIG. 6. However, the image display region 730 is changed to the region 630, and only the operation according to the selection of the target for image processing becomes different therefrom. Accordingly, detailed description is not required here.

Next, the synthesizing process in Step S917 will be described in detail with reference to the flowchart in FIG. 10. The process is a process when the user's indicating position using the mouse 114 (original position of the cursor 650) is located inside the image display region 733.

First, in Step S1001, the signal processing unit 428 reads the OCT cross-sectional image and the IVUS cross-sectional image which are determined in previous Step S916, from the memory 428a.

Next, in Step S1002, coordinates representing the user's indicating position are set as P_ivus and P_oct (refer to FIG. 8).

Thereafter, the process proceeds to Step S1003 to determine whether or not the current mode is the OCT_in_IVUS mode. When it is determined that the current mode is the OCT_in_IVUS mode, the process proceeds to Step S1004.

In Step S1004, the signal processing unit 428 cuts out a partial image inside the circular region having the radius R1 (=R0/M) which is centered on the coordinate P-oct within the OCT cross-sectional image data. Then, the process proceeds to Step S1005 to magnify the cut-out partial image by using the magnification M, and the process proceeds to Step S1006. In Step S1006, the signal processing unit 428 overwrites the obtained magnified partial image inside the circular region having the radius R0, which is centered on the coordinate P_ivus within the IVUS cross-sectional image, and displays the result in the image display region 733. At this time, the images are synthesized to align with the circular frame having the set thickness. However, when the thickness of the circular frame is zero, it is not necessary to synthesize the circular frame.

In accordance with an exemplary embodiment, for example, in Step S1003, when the current mode is not the OCT_in_IVUS mode, that is, when it is determined that the current mode is the IVUS_in_OCT mode, the process can proceed to Step S1007.

In Step S1007, the signal processing unit 428 cuts out a partial image inside the circular region having the radius R1 (=R0/M) which is centered on the coordinate P_ivus within the IVUS cross-sectional image data. Then, the process proceeds to Step S1008 to magnify the cut-out partial image by using the magnification M, and the process proceeds to Step S1009. In Step S1009, the signal processing unit 428 overwrites the obtained magnified partial image inside the circular region having the radius R0, which is centered on the coordinate P_oct within the OCT cross-sectional image, and displays the result in the image display region 733. At this time, the images are synthesized to align with the circular frame having the set thickness. However, when the thickness of the circular frame is zero, it is not necessary to synthesize the circular frame.

Hitherto, the process according to the user interface in the embodiment has been described. According to the magnifier mode in the above-described embodiment, as compared to the parallel display mode, a user can check both the IVUS cross-sectional image and the OCT cross-sectional image by observing the circular frame and the surroundings in conjunction with the mouse 114 operated by the user, thereby enabling the user to easily perform diagnosis on a lesion. Furthermore, the position of the circular frame indicating the magnifier can be freely changed by the user's operation. Accordingly, in the OCT_in_IVUS mode, when the user wants to view the IVUS cross-sectional image of a portion hidden in the OCT cross-sectional image inside the circular frame, the user may view the IVUS cross-sectional image by simply moving the circular frame without changing the user's viewpoint. That is, for the user, the OCT cross-sectional image can be compared with the IVUS cross-sectional image of the user's interest site without changing the user's viewpoint.

A display example described in the above-described embodiment is an example, and the present disclosure is not limited thereto. In particular, for example, a case has been described where the embodiment employs two modes of the OCT_in_IVUS mode and the IVUS_in_OCT mode and any one of these can be designated by the user. However, a configuration of employing only one mode may be adopted. In the latter case, preferably, the cross-sectional image displayed outside the circular frame is the IVUS cross-sectional image, and the cross-sectional image displayed inside the circular frame is the OCT cross-sectional image. The reason is that since the IVUS cross-sectional image enables the user to observe a relatively deep site of biological tissues, it is convenient to display an image in a wide range, and that since the OCT cross-sectional image originally has high resolution, it is possible to sufficiently meet higher magnification.

In addition, as is understood from the above-described embodiment, most of the processes according to the magnifier mode are performed by the signal processing unit 428 configured to include a microprocessor. Accordingly, a function of the microprocessor can be fulfilled by a program causing a computer to execute the process. Therefore, as a matter of course, the program is also included within the scope of the present disclosure. In particular, in the embodiment, the imaging apparatus for diagnosis illustrated in FIG. 1 has been described as an example. However, an application program may cause a general personal computer to execute the process so as to get access to a storage medium (for example, a CD-ROM or a memory card) which stores the IVUS cross-sectional image and the OCT cross-sectional image both of which are obtained by the imaging apparatus for diagnosis illustrated in FIG. 1. As a result, the IVUS cross-sectional image and the OCT cross-sectional image both of which can be read therefrom may be realized as the user interface according to the above-described embodiment. In addition, the program is generally stored in a non-transitory computer-readable storage medium such as the CD-ROM, a DVD-ROM and the like. The program is set to a reading device (CD-ROM drive or the like) included in the computer, and is copied or installed in a system. In this manner, the program can cause the computer to execute the process. Therefore, the related non-transitory computer-readable storage medium is included within the scope of the present disclosure.

The detailed description above describes an imaging apparatus for diagnosis, an information processing apparatus, and a control method. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An imaging apparatus for diagnosis which rotatably and detachably holds a probe having a transceiver in which an ultrasound transceiver for transmitting and receiving ultrasound waves and an optical transceiver for transmitting and receiving light are arranged, and which generates an ultrasound cross-sectional image and an optical cross-sectional image of biological tissues by using the reflected waves reflected from biological tissues and received by the ultrasound transceiver and the reflected light reflected from the biological tissues and received by the optical transceiver, the apparatus comprising:
display means for displaying an image display region for displaying a cross-sectional image, and a user interface including a frame which is located inside the image display region and whose display position is freely changed in accordance with a position instructed by a user; and
display control means for changing the display position of the frame in accordance with the user's instruction, for displaying a first cross-sectional image, one image of the biological tissues between the ultrasound cross-sectional image and the optical cross-sectional image which correspond to each other, in a region outside the frame within the image display region, and for displaying a second cross-sectional image, the other image between the ultrasound cross-sectional image and the optical cross-sectional image, inside the frame.

2. The imaging apparatus for diagnosis according to claim 1, comprising:
changing means for changing a size of the frame.

3. The imaging apparatus for diagnosis according to claim 1, comprising:
magnification setting means for setting magnification of the second cross-sectional image.

4. The imaging apparatus for diagnosis according to claim 3, wherein
the frame is a circular frame, and
when a center position of the circular frame is expressed by a point P (x and y) using coordinates x and y, a radius of the circular frame is set to RO, and the magnification set by the magnification setting means is set to M, the display control means cuts out a partial image inside a circular region centered on the point P (x and y) and having a radius R0/M, from the second cross-sectional image, generates a partial image of a circle having the radius RO by magnifying the cut-out partial image using the magnification M, and displays the magnified partial image inside the circular frame.

5. The imaging apparatus for diagnosis according to claim 1, comprising:
selection means for selecting either a first mode in which the ultrasound cross-sectional image is the first cross-sectional image and the optical cross-sectional image is the second cross-sectional image or a second mode in which the optical cross-sectional image is the first cross-sectional image and the ultrasound cross-sectional image is the second cross-sectional image.

6. The imaging apparatus for diagnosis according to claim 1, comprising:
indicating means for indicating that the frame is visually displayed or for indicating that the frame is not displayed.

7. A control method for an imaging apparatus which rotatably and detachably holds a probe having a transceiver in which an ultrasound transceiver for transmitting and receiving ultrasound waves and an optical transceiver for transmitting and receiving light are arranged, and which generates an ultrasound cross-sectional image and an optical cross-sectional image of biological tissues by using the reflected waves reflected from biological tissues and received by the ultrasound transceiver and the reflected light reflected from the biological tissues and received by the optical transceiver, the method comprising:
a display step of causing display means to display an image display region for displaying a cross-sectional image, and a user interface including a frame which is located inside the image display region and whose display position is freely changed in accordance with a position instructed by a user; and
a display control step of changing the display position of the frame in accordance with the user's instruction, displaying a first cross-sectional image, one image of the biological tissues between the ultrasound cross-sectional image and the optical cross-sectional image which correspond to each other, in a region outside the frame within the image display region, and displaying a second cross-sectional image, the other image between the ultrasound cross-sectional image and the optical cross-sectional image, inside the frame.

8. A program which causes a computer to execute each step of the control method for the imaging apparatus for diagnosis according to claim 7.

9. A non-transitory computer-readable storage medium that stores the program according to claim 8.

10. An information processing apparatus which displays an ultrasound cross-sectional image and an optical cross-sectional image which are obtained by an imaging apparatus for diagnosis for generating the ultrasound cross-sectional image and the optical cross-sectional image, the apparatus comprising:
display means for displaying an image display region for displaying a cross-sectional image, and a user interface including a frame which is located inside the image display region and whose display position is freely changed in accordance with a position instructed by a user; and
display control means for changing the display position of the frame in accordance with the user's instruction, for displaying a first cross-sectional image, one image of the biological tissues between the ultrasound cross-sectional image and the optical cross-sectional image which correspond to each other, in a region outside the frame within the image display region, and for displaying a second cross-sectional image, the other image between the ultrasound cross-sectional image and the optical cross-sectional image, inside the frame.

11. The informational processing apparatus according to claim 10, comprising:
changing means for changing a size of the frame.

12. The informational processing apparatus according to claim 10, comprising:
magnification setting means for setting magnification of the second cross-sectional image.

13. The informational processing apparatus according to claim 12, wherein
the frame is a circular frame, and
when a center position of the circular frame is expressed by a point P (x and y) using coordinates x and y, a radius of the circular frame is set to RO, and the magnification set by the magnification setting means is set to M, the display control means cuts out a partial image inside a circular region centered on the point P (x and y) and having a radius R0/M, from the second cross-sectional image, generates a partial image of a circle having the radius RO by magnifying the cut-out partial image using the magnification M, and displays the magnified partial image inside the circular frame.

14. The informational processing apparatus according to claim 10, comprising:
selection means for selecting either a first mode in which the ultrasound cross-sectional image is the first cross-sectional image and the optical cross-sectional image is the second cross-sectional image or a second mode in which the optical cross-sectional image is the first cross-sectional image and the ultrasound cross-sectional image is the second cross-sectional image.

15. The informational processing apparatus according to claim 10, comprising:
indicating means for indicating that the frame is visually displayed or for indicating that the frame is not displayed.

16. A control method for an information processing apparatus which displays an ultrasound cross-sectional image and an optical cross-sectional image which are obtained by an imaging apparatus for diagnosis for generating the ultrasound cross-sectional image and the optical cross-sectional image, the method comprising:
a display step of causing display means to display an image display region for displaying a cross-sectional image, and a user interface including a frame which is located inside the image display region and whose display position is freely changed in accordance with a position instructed by a user; and
a display control step of changing the display position of the frame in accordance with the user's instruction, for displaying a first cross-sectional image, one image of the biological tissues between the ultrasound cross-sectional image and the optical cross-sectional image which correspond to each other, in a region outside the frame within the image display region, and displaying a second cross-sectional image, the other image between the ultrasound cross-sectional image and the optical cross-sectional image, inside the frame.

17. A program which causes a computer to execute each step of the control method for the information processing apparatus according to claim 16.

18. A non-transitory computer-readable storage medium that stores the program according to claim 17.
